(19) United States
(12) Patent Application Publication
(10) Pub. No.: US 2009/0143388 A1
(43) Pub. Date: Jun. 4, 2009

(54) TREATMENT OF BRAIN DISORDERS

(76) Inventors: Kochupurackal Parameswararnnayer Mohanakumar, Kollam (IN); Goutam Chandra, Dt. Murshidabad (IN); Reena Haobam, Imphal (IN); Michael Lionel Selley, Turramurra (AU)

Correspondence Address: WILSON SONSINI GOODRICH & ROSATI, 650 PAGE MILL ROAD, PALO ALTO, CA, 94304-1050, US

(21) Appl. No.: 12/202,099

(22) Filed: Aug. 29, 2008

Related U.S. Application Data
(60) Provisional application No. 60/968,726, filed on Aug. 29, 2007.

(51) Int. Cl. A61K 31/53; A61P 25/00

(52) U.S. Cl. 514/245

(57) ABSTRACT

Striatal neurodegenerative disorders, e.g. Parkinson's disease, are treated by administration of an effective dose of an antioxidant compound of formula I, formula II or a pharmaceutically acceptable salt thereof, in a regimen that decreases the adverse effects of the disorder. Such adverse effects can motor deficits, which include catalepsy or akinesia. Also provided are kits and systems for practicing the subject methods, as well as methods of use of agents identified in the screening method of the invention.

### BACKGROUND OF THE INVENTION

Parkinson's disease (PD) is an idiopathic, slowly progressive, degenerative CNS disorder characterized by slow and decreased movement, muscular rigidity, resting tremor, and postural instability. The major pathological feature of PD is selective degeneration of dopaminergic neurons in the substantia nigra pars compacta (SNpc) and loss of their terminals in the caudate and putamen. Loss of substantia nigra neurons, which project into the caudate nucleus and putamen, depletes dopamine in these areas. Evidences accumulated in the past indicate that multiple factors, including genetic and environmental ones, contribute to dopaminergic neurodegeneration in this neurodegenerative disease.

Diagnosis is clinical. Parkinson's disease is suspected in patients with characteristic resting tremors, decreased movement, or rigidity. Diagnosis is confirmed by the presence of other characteristic signs, such as infrequent blinking, lack of facial expression, impaired postural reflexes, and/or characteristic gait abnormalities. Tremor without other characteristic signs suggests early disease or another diagnosis.

Currently available treatment for PD traditionally starts with levodopa. However, some experts believe that early use of levodopa hastens development of adverse effects and failure of drug response; they prefer to withhold if possible and use anticholinergic drugs, amantadine, or dopamine agonists first. Dopamine agonists directly activate dopamine receptors in the basal ganglia. They can be used as monotherapy but, as such, are rarely sufficient for more than a few years. Dopamine agonists are particularly useful in later stages when response to levodopa decreases or on-off effects are prominent. Anticholinergic drugs can be used as monotherapy in early disease and later to supplement levodopa. If drugs are ineffective and disease is advanced, surgery is considered; high-frequency electrical stimulation of the subthalamic nucleus is the treatment of choice. For patients with severe tremor, deep brain stimulation of the ventral intermediate nucleus in the thalamus may help. Transplantation of fetal dopamine neurons is an experimental treatment that is hoped to replace dopamine in the brain.

Injection of the neurotoxin, 1-methyl-4-phenyl-1,2,3,6-tetrahydropyridine (MPTP) is known to cause clinical symptoms similar to sporadic PD. Since humans, non-human primates and rodents are quite susceptible to this neurotoxin, administration of MPTP is one of the most common methods to develop animal models for investigating the pathological mechanisms of the disease and for drug screening against PD (see Beal (2001) Nat. Rev. Neurosci. 2, 325-334.) Many of the initial syndromes exhibited following MPTP administration have been well characterized, and swim inability has been shown to be directly correlated with severity of striatal DA depletion and motor dysfunction (Haobam et al. (2005) Behav. Brain Res. 163, 159-167).

A number of mechanisms, including mitochondrial dysfunctions and generation of reactive oxygen radicals leading to oxidative stress, have been postulated for the dopaminergic neurodegeneration characteristic of PD (Thomas et al. (2000) Brain Res. 852, 221-224). Besides mechanisms intrinsic to dopaminergic neurons, cell-cell interactions associated with inflammatory reactions may also have important role in PD pathogenesis (see Herrera et al. (2005) J. Neural Transm. 112, 111-119). A large number of reactive microglia are found in the SN region of the postmortem PD brain samples and in mouse brain after MPTP insult. Experimental microglial activation by lipopolysaccharide or thrombin leads to degeneration of midbrain dopaminergic neurons in vitro (see Katsuki et al. (2006) J. Neurochem. 97:1232-1242) and in vivo (Carreno-Muller et al. (2003) J. Neurochem. 84:1201-1214.).

PD prominently features dopamine transmitter insufficiency, and current management is almost exclusively reliant on dopamine replacement drugs. But, while these drugs are initially effective in most patients, they do not slow the underlying degeneration in the area of the brain most affected, the substantia nigra (SN). Their effectiveness declines over time and their adverse effects become increasingly more troublesome. Broader options for long-term management are urgently needed.

### Publications

Hori et al., Jpn. J. Cancer Res. 1997; 88:12-17; and U.S. Pat. No. 3,966,728 to Nippon Shinyaku, herein specifically incorporated by reference including reference to teachings related to methods of preparation and specific compounds.

### SUMMARY OF THE INVENTION

The invention comprises compositions and methods for the treatment of neurodegenerative disorders. The methods of present invention can be useful for the protection of neurons from molecular events adversely affecting the survival of neurons.

In one embodiment, for example, composition and the methods can be effective for preventing the loss of striatal neurons. Examples of neurodegenerative disorders that can be treated or prevented by the methods and compositions of the invention include Parkinson's disease and Huntington's disease, particularly Parkinson's disease, and animal models thereof.

Other disorders that can be treated or prevented using the present invention disorders having Parkinson-like symptoms, disabilities stemmed from the loss, inactivity or decreased activity of D2 dopaminergic neurons, disabilities suspected from striatal abnormalities such as attention deficit disorder, REM sleep disorder, or other cognitive disorders associated with reduced activity of striatal neurons in the brain.

In one aspect of the invention, a method is provided for treating, reducing or preventing a neurodegenerative disorder comprising administering to a subject in need thereof in a therapeutically effective amount a composition of one or more antioxidant disclosed herein. In one embodiment, the antioxidant is a compound of formula 1 or a pharmaceutically acceptable salt thereof:

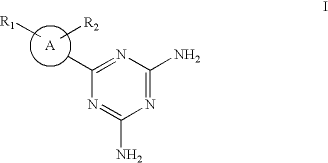

where A is an aromatic ring of 5 or 6 carbons, optionally substituted on an annular carbon with N, S or O;

R₁ and R₂ are independently selected from H, F, Cl, I, Br, C1-6 alkoxy or substituted alkoxy groups, where substituents are selected from amino or substituted amino wherein the substituents are 1-4 carbon alkyl, 3-6 carbon cycloalkyl, or the amino group may be part of a ring containing 3-6 carbon atoms. R₁ and R₂ may be in the meta, para or ortho position relative to each other, preferably para.

In other embodiments, the antioxidant is a compound of Formula II or a pharmaceutically acceptable salt thereof:

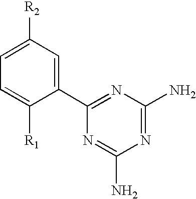

where R₁ and R₂ are independently selected from H, F, Cl, I, and Br.

In yet other embodiments the antioxidant is irsogladine, 2,4-diamino-6-(2,5-dichlorophenyl)-s-triazine or a pharmaceutically acceptable salt thereof.

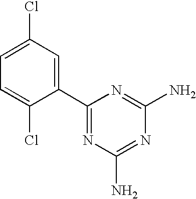

In one aspect of the invention, a method for treatment of a neurodegenerative disorder comprises administration of one or more antioxidants of the invention and administration of one or more additional therapeutic agents. In further embodiments, the one or more additional therapeutic agent is administered before, concurrent with, or after administration of one or more antioxidants of the invention.

In one embodiment, the invention provides a method of preventing or treating catalepsy or akinesia in a subject suffering from a striatal neurodegenerative disorder or predisposed to develop a striatal neurodegenerative disorder, the method comprising administering an effective dose of an antioxidant of formula I or Formula II to the subject in a regimen that decreases catalepsy or akinesia.

In another embodiment the invention provides a method of preventing or treating striatal dopamine depletion in a subject suffering from striatal dopaminergic neurodegeneration, the method comprising administering an effective dose of an antioxidant of formula I or formula II to the subject in regimen that decreases dopamine depletion. Dopamine depletion can be indirectly measured by a depletion of dopamine metabolites, e.g. 3,4-dihydroxyphenylacetic acid and homovanillic acid, which depletion is prevented or reversed by the methods of the invention.

In another embodiment the invention provides a method of attenuating production of hydroxyl radicals in mitochondria of relevant, i.e. striatal, neurons in a subject suffering from a striatal neurodegeneration disorder, the method comprising administering an effective dose of an antioxidant of formula I or formula II to the subject in regimen that attenuates production of hydroxyl radicals in mitochondria of relevant neurons.

In some embodiments of the invention the subject is a human. In other embodiments of the invention, the subject is a non-human subject, e.g. a laboratory animal including rats, mice, etc. In such animals, a striatal neurodegenerative disorder may be experimental induced, e.g. by the administration of MPTP, by genetic or environmental manipulation, and the like.

Another aspect of the present invention is directed to the use of an antioxidant of formula I or formula II or a physiologically acceptable salt thereof in the manufacture of a medicament for the treatment of a striatal neurodegenerative disorder. Yet another aspect is directed to the use an antioxidant of formula I or a physiologically acceptable salt thereof in the manufacture of a medicament for the treatment of a striatal dopaminergic neurodegenerative disorder. Such a disorder includes Parkinson's disease.

Yet another aspect of the present invention relates to the antioxidants of formula I or formula II or a physiologically acceptable salt thereof as hereinbefore defined, when used in the methods of the present invention.

These and other aspects of the present invention, various embodiments of the invention and methods for making and using the invention are described in more detail in the description of the drawings and the invention, the examples, the claims, and the drawings that follow.

### BRIEF DESCRIPTION OF THE DRAWINGS

FIG. 1. Irsogladine scavenges MPP⁺-induced OH from the mitochondria. Sub-mitochondrial particles were incubated with the neurotoxic metabolite of MPTP, 1-methyl-4-phenyl pyridinium ion (MPP⁺) and salicylic acid in the presence and absence of irsogladine. The resulting OH adducts of salicylate, 2,3-dihydroxy benzoic acid (2,3-DHBA) was assayed using a HPLC equipped with electrochemical detector. MPP+ caused about 5-fold increase in OH production in the mitochondria, which was dose-dependently scavenged by irsogladine. *Significantly reduced from the MPP⁺ value; p±0.05; Results given are mean±S.E.M., n=4.

FIG. 2. Irsogladine reverses MPTP-induced reductions in striatal DA metabolism. Striatal DA (A) and its metabolites, DOPAC (B) and HVA (C) were assayed employing a sensitive HPLC-electrochemical procedure on the 5th day following the administration of MPTP (30 mg/kg; administered twice, 16 h apart, i.p.). Irsogladine (25, 50 and 100 mg/kg, p.o.) was administered for two days at 12 h intervals following the second MPTP injection. Results given are mean±S.E.M., n=6-8 for control groups and 10-12 for MPTP-treated groups (*p<0.05 vs. control; Student's t-test).

FIG. 3. Effect of irsogladine on MPTP-induced akinesia (A) and catalepsy (B). Assessment of catalepsy was undertaken 2 h after the treatment of MPTP in Balb/c mice. Animals that received MPTP showed maximum latency (in seconds) to move all the four limbs (akinesia; Fig. A) and time (in seconds) to change an externally imposed posture (catalepsy; Fig. B). Irsogladine administration dose-dependently reduced both the behavioral disabilities. Results given are mean±S.E.M., n=6-8 for control group and 10-12 for MPTP-treated groups (*p<0.01 vs. control; Mann-Whitney Rank Sum test).

FIG. 4. Irsogladine attenuates MPTP-induced swim disability in mice. Effect of different doses of Irsogladine on the swimming ability of Balb/c mice was tested in warm water (27±2° C.) on the fourth day following the treatment of MPTP (30 mg/kg, i.p., twice, 16 h apart). Swim-scores were recorded on a performance intensity scale of 0-3 for all the animals for 10 min. Results given are mean±S.E.M., n=6-8 for control groups and 10-12 for MPTP-treated groups (p≦0.05; vs. control; Wilcoxon Signed Rank test for group comparison).

### DETAILED DESCRIPTION OF THE EMBODIMENTS

Striatal neurodegenerative disorders, e.g. Parkinson's disease, are treated by administration of an effective dose of an antioxidant compound of formula I or formula II or a pharmaceutically acceptable salt thereof, in a regimen that decreases the adverse effects of the disorder. Such adverse effects can be motor deficits, which include catalepsy or akinesia.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an individual” includes one or more individuals and reference to “the method” includes reference to equivalent steps and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In the formulas provided herein, molecular variations are included, which may be based on isosteric replacement. “Isosteric replacement” refers to the concept of modifying chemicals through the replacement of single atoms or entire functional groups with alternatives that have similar size, shape and electromagnetic properties, e.g. O is the isosteric replacement of S, N, COOH is the isosteric replacement of tetrazole, F is the isosteric replacement of H, sulfonate is the isosteric replacement of phosphate etc.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

As used herein, “suitable conditions” for carrying out a synthetic step may be discerned by reference to publications directed to methods used in synthetic organic chemistry. The reference books and treatise set forth above that detail the synthesis of reactants useful in the preparation of compounds of the present invention will also provide suitable conditions for carrying out a synthetic step according to the present invention.

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, maleate, procaine, hydrabamine, choline, betaine, ethylenediamine, glucosamine, methylglucamine, theobromine, purines, piperazine, piperidine, N-ethylpiperidine, polyamine resins and the like.

The antioxidants, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The present invention provides antioxidants in a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the antioxidants is achieved in various ways, although oral administration is a preferred route of administration. In some formulations, the antioxidants are systemic after administration; in others, the inhibitor is localized by virtue of the formulation, such as the use of an implant that acts to retain the active dose at the site of implantation.

In some pharmaceutical dosage forms, the antioxidants are administered in the form of their pharmaceutically acceptable salts. In some dosage forms, the antioxidant is used alone, while in others, the antioxidant is used in combination with another pharmaceutically active compounds, e.g. levodopa. The following methods and excipients are merely exemplary and are in no way limiting.

For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

The active compounds can be incorporated into a variety of formulations for therapeutic administration. Part of the total dose may be administered by different routes. Such administration may use any route that results in systemic absorption, by any one of several known routes, including but not limited to inhalation, i.e. pulmonary aerosol administration; intranasal; sublingually; orally; and by injection, e.g. subcutaneously, intramuscularly, etc.

For injectables, the agents are used in formulations containing cyclodextrin, cremophor, DMSO, ethanol, propylene glycol, solutol, Tween, triglyceride and/or PEG. For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of antioxidants calculated in an amount sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In another aspect of the invention, one or more antioxidants of the invention are combined with one or more additional therapeutic agents useful in treating or preventing a neurodegenerative disorder. As used herein in the context of combination treatment, the term “combined” or “combination” means one or more antioxidants of the invention are administered concurrent with, prior to or subsequent to one or more additional therapeutic agent or therapeutic regimen (e.g., surgery).

In one embodiment, one or more antioxidant of the invention is administered in combination with one or more phosphodiesterase inhibitors. Suitable phosphodiesterase inhibitors include but are not limited to vinpocetine, EHNA, enoximone, milrinone, mesembrine, rolipram, ibudilast, sidenafil, vardenafil, avanafil, udenafil, or dipyridamole

In various embodiments, one or more antioxidants of the invention are used in combination with a steroidal anti-inflammatory to treat or prevent a neurodegenerative disorder. Suitable steroidal anti-inflammatory agents include, but are not limited to, hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures of two or more of the foregoing.

In another embodiment, one or more antioxidants of the invention are used in combination with a non-steroidal anti-inflammatory to treat or prevent a neurodegenerative disorder. Suitable non-steroidal anti-inflammatory agents, include, but are not limited to, 1) the oxicams, such as piroxicam, isoxicam, tenoxicam, and sudoxicam; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

In yet another embodiment, one or more antioxidants of the invention are used in combination with one or more anti-inflammatory to treat a neurodegenerative disorder. Suitable anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; -Dexamethasone Dipropionate; Diclofenac Potassium; Diclofenac Sodium; Diflorasone Diacetate; -Diflumidone Sodium; Diflunisal; Difluprednate; Diftalone; Dimethyl Sulfoxide; Drocinonide; Endrysone; Enlimomab Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

In yet another embodiment, one or more antioxidants of the invention are used in combination with one or more serotonin receptor inhibitors (SSRIs) and other anti-depressants, anxiolytics (e.g. alprazolam), etc. Anti-depressants include, but are not limited to, serotonin reuptake inhibitors, such as Celexa, Desyrel, Effexor, Luvox, Paxil, Prozac™, Zoloft, and Serzone; tricyclics, such as Adapin, Anafrinil, Elavil, Janimmine, Ludiomil, Pamelor, Tofranil, Vivactil, Sinequan and Surmontil; monoamine oxidase inhibitors such as Eldepryl, Marplan, Nardil and Parnate. Anti-anxiety agents include, but are not limited to, azaspirones, such as BuSpari; benzodiazepines such as Ativan, Librium, Tranxene, Centrax, Klonopin, Paxipam, Serax, Valium and Xanax and beta-blockers, such as Inderal and Tenormin,

In some embodiments, one or more antioxidants of the invention are used in conjunction with Levodopa which has been an effective treatment for motor symptoms, such as Parkinson's disease.

In another embodiment, one or more antioxidants of the invention are used in combination with surgical treatment to treat a neurodegenerative disorder. For example, in one embodiment, surgical treatment is used in late-stage management of Parkinsons' Disease. In further embodiments, one or more antioxidants of the invention are used in conjunction with therapeutic agents used to treat PD. Currently, five classes of drugs comprise the pharmacological armamentarium for the motor symptoms of PD. Dopaminergic agents include Levodopa/carbidopa. Dopamine agonists include Apomorphine (Apokyn®), Bromocriptine (Parlodel®), Cabergoline, Lisuride, Pergolide (Permax®), Pramipexole (Mirapex®), and Ropinirole (Requip®). COMT inhibitors include Entacapone and Tolcapone. MAO-B inhibitors include Rasagiline (Azilect®) Selegiline (Eldepryl®, Zelapar®). Anticholinergics include Trihexyphenidyl (Artane®), Benztropine and Ethopropazine. Amantadine (Symmetrel®) also finds use. These agents may be administered in combination with the antioxidants of the present invention.

Therapeutic agent(s) or a subject can be assessed to determine a treatment regimen or combination of therapeutic agents for treatment or prevention of a neurodegenerative disorder. The term “assess”, “assaying”, “assessed” or “assessing” includes any form of measurement, and includes determining if an element is present or not. The terms “determining”, “measuring”, “evaluating”, “assess”, “assessed”, “assessing” and “assaying” are used interchangeably and include quantitative and qualitative determinations. Assessing may be relative or absolute. “Assessing the presence of” includes determining the amount of something present, and/or determining whether it is present or absent. In the methods of the invention, an effective dose, or effective regimen, is a combination of dose and dosing that provides for an improvement in the symptoms associated with the disease, e.g. as assessed by UPDRS, or the use of surrogate markers as described below. For example, the motor abilities of a patient may improve, where motor symptoms may include motor fluctuations, dyskinesias, off-period dystonia, freezing, and falls. Alternatively, improvement may be assessed by imaging, e.g. by monitoring of dopamine uptake, or striatal neuron function.

The standard tool for tracking Parkinson's disease progress and response to therapy is the United Parkinson's Disease Rating Scale (UPDRS). The UPDRS is subdivided into three scales including cognitive and mood aspects, motor aspects, and activities of daily living (ADL). A lower score indicates a better condition than a higher score. The UPDRS is readily available, e.g. see Fahn S, Elton R, Members of the UPDRS Development Committee. In: Fahn S, Marsden C D, Caine D B, Goldstein M, eds. Recent Developments in Parkinson's Disease, Vol 2. Florham Park, N.J. Macmillan Health Care Information 1987, pp 15 3-163, 293-304.

Other endpoints for determining an effective dose of an agent are available to those of skilled in the art. Examples of endpoints which find use in dosage determination include the time to reach a disease milestone in untreated PD patients (ie, need for levodopa); the change in motor score between initial visit and final visit after washout of all study medications; surrogate markers directed to the integrity of nigrostriatal function, such as striatal uptake of fluorodopa on positron emission tomography (PET), or beta-CIT-on single-photon emission computerized tomography (SPECT) (see Stocchi and Olanow (2003) Ann Neurol. 53 Suppl 3:S87-97). Other surrogate endpoints are discussed by Biglan and Holloway (2003) Curr. Neur. and Neuroscience Reports 3:314-320. The use of [¹²³I]β-CIT and SPECT imaging of the dopamine transporter as a biomarker of PD onset and severity is disclosed by Marek et al. (2001) Neurology 57:2089-2094.

The terms “reference” and “control” are used interchangeably to refer to a known value or set of known values against which an observed value may be compared. As used herein, known means that the value represents an understood parameter.

As used herein, “treatment” or “treating” refers to inhibiting the progression of a disease or disorder, e.g., Parkinson's disease, or delaying the onset of a disease or disorder, e.g., Parkinson's disease, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. With respect to Parkinson's disease, effective treatment may address the adverse symptoms of the disease, e.g. akinesia, catalepsy, etc., without altering the progression of the disease. The effect may be prophylactic in terms of completely or partially preventing a symptom, disease or condition. “Treatment,” as used herein, covers any treatment of a disease or disorder in a subject, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of symptoms associated with Parkinson's disease.

Therapeutic agents. In the methods of the invention, an effective dose of one or more antioxidant compound of formula I or formula II as described above, or a pharmaceutically acceptable salt thereof is provided to a subject suffering from a neurodegenerative disorder.

In some embodiments, the one or more antioxidant is administered in combination with one or more additional therapeutic agent, as described above, for treating or preventing a neurodegenerative disorder. In one embodiment, the neurodegenerative disorder is a striatal neurodegenerative disorder. In a further embodiment, the disorder is Parkinson's disease.

In a further embodiment, administration of antioxidants (or in combination with one or more additional therapeutic agent) is in a regimen that decreases the adverse effects of a neurodegenerative disorder.

In some embodiments of the invention, the therapeutic agent is irsogladine, 2,4-diamino-6-(2,5-dichlorophenyl)-s-triazine, or a salt thereof, e.g. a maleate salt. Irsogladine has been approved as a drug for the treatment of ulcers, and has been reported to prevent the gastric mucosal damage in several experimental animal models. It has a number of pharmacological activities, including antioxidant activity, and inhibition of phosphodiesterase IV (PDE IV). Irsogladine is shown herein to dose-dependently reduced the depletion of striatal DA, DOPAC and HVA in an animal model for degeneration of dopaminergic neurons, and to reduce adverse motor symptoms.

### Methods of Use

A therapeutic regimen of an antioxidant of formula I or formula II is administered to a subject suffering from a striatal neurodegenerative disorder. Administration may be topical, localized or systemic, depending on the specific disease and agent. In some embodiments, e.g. where the agent is irsogladine, administration can be oral. The compound is administered at an effective dosage that over a suitable period of time reduces the motor deficits associated with striatal brain disorders, while minimizing any side-effects. It is contemplated that the composition will be obtained and used under the guidance of a physician for clinical use.

The efficacy of a particular drug and dose may be determined by in vitro testing or in vivo testing. The dose will vary depending on the specific compounds utilized, patient status, etc., at a dose sufficient to improve patient mobility, while otherwise maintaining patient health.

Depending on the subject and condition being treated and on the administration route, the active compounds are administered in dosages of around about 0.1 mg to 2000 mg/kg body weight per day, e.g. about 100, about 500, about 1000, about 10,000 mg/day for an average person. Durations of the regimen may be from: 1×, 2×, 3× daily. Some of the inhibitors of the invention are more potent than others. Preferred dosages for a given inhibitor are readily determinable by those of skill in the art by a variety of means. A preferred means is to measure the physiological potency of a given compound.

Various methods for administration are employed in the practice of the invention. The dosage of the therapeutic formulation can vary widely, depending upon the nature of the disease, the frequency of administration, the manner of administration, the clearance of the agent from the patient, and the like. The initial dose can be larger, followed by smaller maintenance doses. The dose can be administered as infrequently as weekly or biweekly, or more often fractionated into smaller doses and administered daily, with meals, semi-weekly, and the like, to maintain an effective dosage level.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Experimental

### Irsogladine Protects Against Striatal Dopamine Depletion and Behavioral Abnormalities

### Results

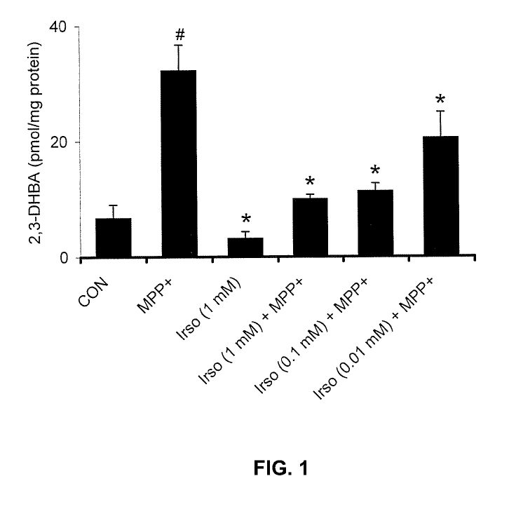

Irsogladine scavenges MPP⁺-induced hydroxyl radical (.OH) in the mitochondria. The oxidative metabolite of MPTP, 1-methyl-4-phenylpyridinium ion (MPP⁺) is selectively taken into the dopaminergic neurons through dopamine transporters, and sequestered in the mitochondria. Incubation of sub-mitochondrial particles with MPP⁺ resulted in 5-fold increase in the production of 2,3-dihydroxybenzoic acid (2,3-DHBA), the hydroxyl radical adduct of salicylic acid. Irsogladine significantly attenuated 2,3-DHBA production in the mitochondria dose dependently (0.01-1 mM; FIG. 1).

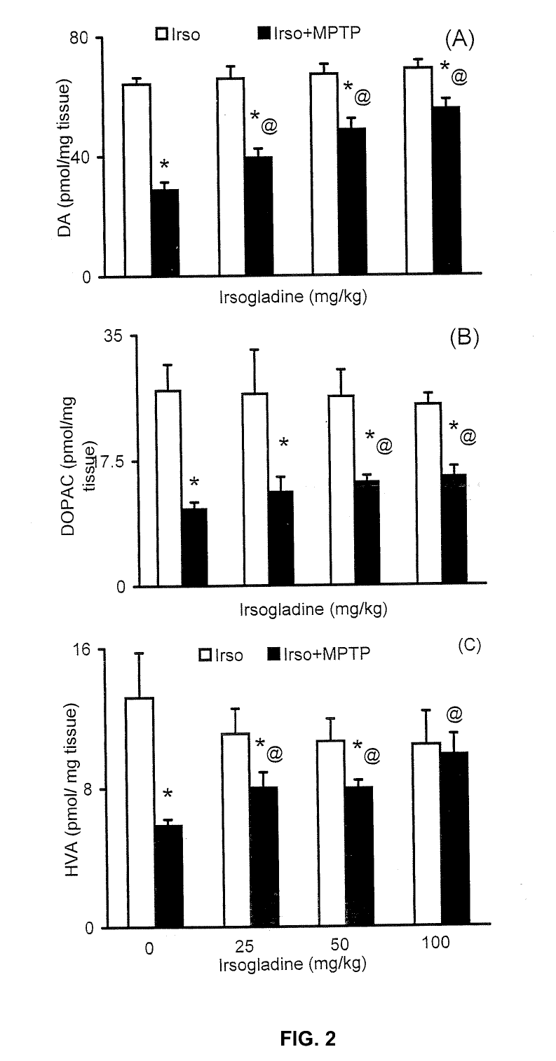

Irsogladine protects against MPTP-induced striatal DA depletion. MPTP administration (30 mg/kg; i.p., 2 times 16 h apart) caused 55% depletion of striatal DA on the 5^{th} day compared to saline-injected mice (FIG. 2A). Irsogladine post-treatment (25, 50 and 100 mg/kg p.o., b.i.d. for 2 days) in these striatal denervated mice elicited significant dose-dependant protection of striatal DA (FIG. 2A). Irsogladine treated singularly did not cause significant alteration of striatal DA in control animals.

Irsogladine post-treatment in MPTP-treated mice also caused dose-dependant reversal of DA metabolites, 3,4-dihydroxyphenylacetic acid (DOPAC; FIG. 2B) and homovanillic acid (HVA; FIG. 2C). Irsogladine treatment alone did not cause any significant alterations in the levels of these metabolites (FIG. 2B,C). The levels of serotonin (5-HT) and its metabolite, 5-hydroxyindoleacetic acid (5-HIAA) were not altered in animals treated with MPTP and/or Irsogladine (Table 1).

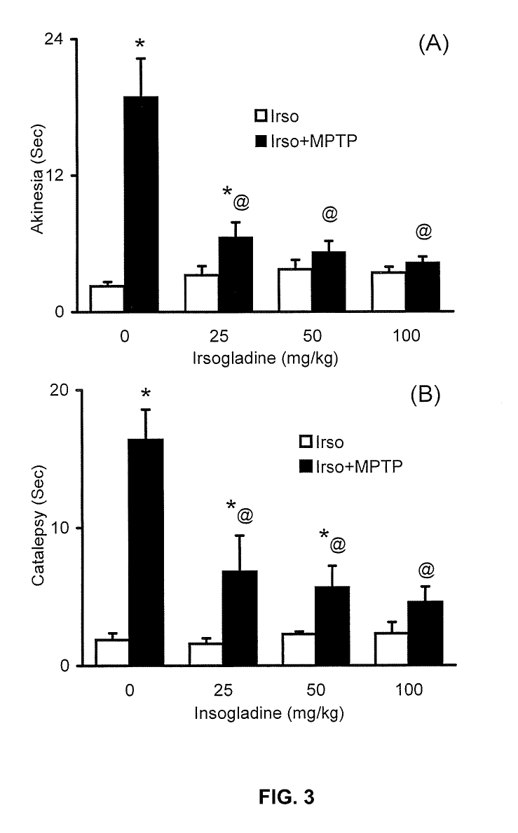

Attenuation of MPTP-induced akinesia and rigidity by irsogladine. Two hours after the second injection of MPTP or saline, motor functions of the animals were evaluated. MPTP-treated animals displayed significant akinetic (FIG. 3A) and cataleptic (FIG. 3B) behaviors compared to saline treated mice. Irsogladine administration dose-dependently attenuated these effects in MPTP treated mice, which was complete for the highest dose of the drug (FIG. 3A,B). Akinesia and rigidity were absent in animals treated with irsladine alone (FIG. 3A,B).

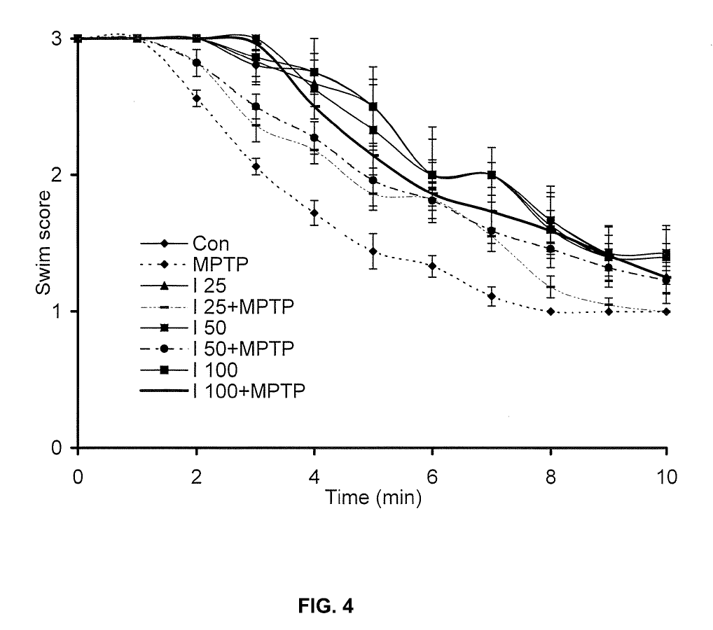

Irsogladine improves MPTP-induced reduction of swim-ability in animals. The swim ability of the animals was measured on the 4^{th} day. MPTP-treated animals showed significant reduction of swim score as compared to sham-treated animals from the 2nd minute onwards (FIG. 4). Irsogladine post-treatment significantly improved the swim score in these animals dose-dependently (FIG. 4).

TABLE 1
Effect of irsogladine and/or MPTP
on striatal 5-HT and 5-HIAA levels

| | 5-HT (pmol/ | 5-HIAA (pmol/ |
|---|---|---|
| | mg tissue; | mg tissue; |
| Treatment | Mean ± SEM) | Mean ± SEM) |
| Control | 2.73 ± 0.38 | 2.57 ± 0.19 |
| MPTP | 2.77 ± 0.31 | 2.50 ± 0.24 |
| Irsogladine (25 mg/kg) | 2.52 ± 0.34 | 2.32 ± 0.31 |
| Irsogladine (25 mg/kg) + MPTP | 2.64 ± 0.24 | 2.11 ± 0.17 |
| Irsogladine (50 mg/kg) | 2.96 ± 0.61 | 2.99 ± 0.29 |
| Irsogladine (50 mg/kg) + MPTP | 2.97 ± 0.27 | 2.88 ± 0.17 |
| Irsogladine (100 mg/kg) | 2.52 ± 0.28 | 2.32 ± 0.16 |
| Irsogladine (100 mg/kg) + MPTP | 2.68 ± 0.24 | 2.01 ± 0.09 |

Serotonin (5-HT) and its metabolite were assayed employing a sensitive HPLC-electrochemical procedure in the striata of Balb/c mice on 5th day following MPTP treatment (30 mg/kg; administered twice, 16 h apart, i.p.). Irsogladine (25, 50 and 100 mg/kg, p.o.) was administered for two days at 12 h intervals following second MPTP injection. Results given are mean±S.E.M., n=6-8 for control groups and 10-12 for MPTP-treated groups

### Discussion

The salient features of the present study are: (i) irsogladine treatment significantly reduced severity of the akinesia and catalepsy in mice following the treatment of the parkinsonian neurotoxin, MPTP, (ii) the drug also improved the swim-ability of these parkinsonian animals, and (iii) it dose-dependently protected against MPTP-induced depletion of striatal DA and its metabolites, DOPAC and HVA. This is the first demonstration of irsogladine's ability to scavenge OH produced in the mitochondria by the parkinsonian neurotoxin, MPP⁺. This is also the first report on the neuroprotective abilities of this drug in PD.

The behaviors such as akinesia and catalepsy that we studied in MPTP-administered mice are largely transient, acute effects, except the swim ability, which can be directly related to dopamine deficiency in the nigrostraiatal system. Assessment of subtle behaviors caused by moderate doses of MPTP has suggested involvement of dopamine transporter, vesicular monoamine transporter, and tyrosine hydroxylase expression, all of which are striatal dopamine related parameters. However, it has been argued that there is involvement of adenosinergic (specifically adenosine A_{2A}), serotoninergic (5-HT_{1A}) or GABA-ergic (GABA-B receptors) in the reversal of MPTP-induced catalepsy or akinesia in mice.

Certain phosphodiesterase inhibitors that have been investigated for neuprotective uses have also been reported to have antipsychotic activities, and could be associated with catalepsy (see Siuciak et al. (2006) Neuropharmacology 51, 386-396; Kanes et al. (2007) Neuroscience 144, 239-246). The data provided herein demonstrate that irsogladine does not cause catalepsy. The antagonistic effect of irsogladine on MPTP-induced catalepsy further indicates its potential as an antiparkinsonian agent.

The present findings indicate that irsogladine is an ideal drug candidate for PD. Irsogladine not only protects against striatal DA depletion, but also many of the MPTP-induced behavioral dysfunctions.

### Experimental Procedures

Drugs and chemicals. MPTP-HCl, MPP⁺, DA, DOPAC, HVA, 5-HIAA, 5-HT, 2,3-DHBA, sodium salicylate and ethylene diamine tetra acetic acid di-sodium salt were purchased from Sigma-Aldrich Co. St. Louis, Mo., USA. Irsogladine was obtained from Shanghai Rory Fine Chemicals Co. Ltd., Shanghai, China. Heptane sulphonic acid and acetonitrile were purchased from SISCOResearch Laboratories (Mumbai, India). High viscosity carboxymethyl cellulose (CMC) sodium salt was purchased from Hi-Media Laboratories, Mumbai, India. Water was distilled in quartz distillation apparatus and deionized using a TKA LAB MICRO high purity water system (TKA, Niederelbert, Germany). All other chemicals were of analytical grade and were procured locally.

Animals. Adult male Balb/c mice, weighing 20-25 g were used in the present study. They were housed under standard conditions of temperature (22±1° C.), humidity (60±5%) and illumination (12-h light/dark cycle). The experimental protocol met the National Guidelines on the ‘Proper Care and Use of Animals in Laboratory Research’ (Indian National Science Academy, New Delhi, 2000) and was approved by the Animal Ethics Committee of the Institute. The procedures adhered to the NIH Guidelines for the Care and Use of Laboratory Animals.

Experimental design. The animals were divided into eight groups. The first four groups of animals received either vehicle (0.5% CMC) or different doses of irsogladine (25, 50, and 100 mg/kg). The fifth group of animals was administered with MPTP. The remaining three groups received the two doses of MPTP, and different doses of irsogladine (25, 50, and 100 mg/kg). Each of the control groups consisted of 6-8 animals; while MPTP treated groups consisted of 10-12 animals. The total period of study was 5 days. Each experiment was repeated at least twice on separate days.

MPTP was dissolved in normal saline and administered intraperitonially (i.p.), twice, 16 h apart, at the dose of 30 mg/kg to mice. The animals received the first injections at 1700 h and the second at 0900 h the next day. Different doses of irsogladine (25, 50, and 100 mg/kg) were suspended in 0.5% sodium CMC and administered to control and MPTP-treated mice. Irsogladine administration started 30 min after the second MPTP injection and continued at an interval of 12 h for the next two days. The volume of injection was 10 μl/g body weight.

### Behavior

Akinesia. Akinesia was measured by noting the latency in seconds (s) of the animals to move all four limbs and the test was terminated if the latency exceeded 180 s. Each animal was initially acclimatized for 5 min on a wooden elevated platform (40 cm×40 cm×30 cm) used for measuring akinesia in mice. Using a stopwatch, the time taken (s) by the animal to move all the four limbs was recorded. This exercise was repeated five times for each animal.

Catalepsy. The term implies the inability of an animal to correct an externally imposed posture. Catalepsy was measured by placing the animals on a flat horizontal surface with both the hind limbs on a square wooden block (3 cm high) and the latency in seconds was measured to move the hind limbs from the block to the ground. The animals were initially acclimatized on the wooden platform.

Swim-ability. Swim-test was carried out on 4^{th} day after MPTP treatment in water tubs (40 cm×25 cm×16 cm). The depth of water was kept at 12 cm and the temperature was maintained at 27±2° C. The animals were wiped dry immediately after the experiment using a dry towel and returned to cages kept at 27±2° C. Swim-score scales were: 0, hind part sinks with head floating; 1, occasional swimming using hind limbs while floating on one side; 2, occasional floating/swimming only; 3, continuous swimming (see Haobam et al. (2005) Behav. Brain Res. 163, 159-167).

Analysis of biogenic amines by HPLC-electrochemistry. DA, DOPAC, HVA, 5-HT, and 5-HIAA were assayed employing HPLC equipped with an electrochemical detector. Animals were sacrificed on the 5^{th} day by cervical dislocation. Striata were quickly dissected out from the left and right hemispheres of the brain, weighed, sonicated in chilled HClO₄ (0.1 M) containing 0.05% EDTA and centrifuged at 12,500×g for 5 min. The supernatant was injected into an HPLC-ECD system (Merck-Hitachi, Germany). The biogenic amines were separated on an ion-pair Ultrasphere RP analytical column (Beckmann, Calif.). The mobile phase contained 8.65 mM heptane sulfonic acid, 0.27 mM EDTA, 13% acetonitrile (HPLC grade), 0.45% triethylamine, and 0.25% phosphoric acid (v/v). The flow rate was 0.7 ml/min and the glassy carbon working electrode was kept at 0.74 V.

Preparation of sub-mitochondrial particles and estimation of hydroxyl radical. Mitochondrial P₂ fraction was obtained by making a 10% homogenate of the cerebral cortex, with a glass-Teflon homogenizer in 0.32 M sucrose in cold 10 mM potassium phosphate buffer, pH 7.2. The homogenate was centrifuged at 1,000×g for 10 min at 4° C. using a Sorvall centrifuge. The supernatant was centrifuged at 10,000×g for 30 min. The pellet was resuspended in the same volume of ice-cold 50 mM Tris in 10 mM potassium phosphate buffer (pH 7.2) and centrifuged at 10,000×g for 30 min. This step was repeated once. The pellet thus obtained was suspended in the same volume of ice-cold 10 mM potassium phosphate buffer, pH 7.2 and kept overnight at −20° C. The mitochondrial suspension was thawed, and the protein content was estimated.

The .OH adduct of salicylate, 2,3-dihydroxybenzoic acid (DHBA), was assayed employing HPLC-electrochemistry as described earlier. The ex-vivo OH production was tested in sub-mitochondrial particles. Mitochondrial fraction was incubated with MPP⁺ (100 μM) and phosphate buffer (0.1 M) for 5 min. Sodium salicylate (0.75 mM) and different concentrations of irsogladine (0.01-1 mM) were added 5 min after the addition of MPP⁺ and incubated for 30 min. The reaction was stopped by adding equal amount of ice-cold perchloric acid (0.1 M) containing 0.01% EDTA, kept on ice for 30 min and then centrifuged at 10,000×g for 5 min. The supernatant was injected into the HPLC system for the assay of 2,3-DHBA. Standard solution containing 2,3-DHBA was injected prior to and after the analyses of the samples, and the products formed are expressed as pmol/mg protein/min.

Statistical analysis. Student's t-test was used for finding significant differences between two means in the neurochemical assays. The data for behavioural studies were statistically evaluated for significance employing non-parametric analyses, Wilcoxon Signed Rank test (for swim-ability) and Mann-Whitney Rank Sum test (for akinesia and catalepsy) employing statistical package, Sigmastat 3.0. Results are given as mean±S.E.M. values. Values of p<0.05 were considered statistically significant.

Abbreviations. 1-Methyl-4-phenyl-1,2,3,6-tetrahydropyridine, MPTP; 1-Methyl-4-phenylpyridinium, MPP⁺; 3,4-Dihydroxyphenylacetic acid, DOPAC; 5-Hydroxyindoleacetic acid, 5-HIAA; Carboxymethyl cellulose, CMC; Dihydroxybenzoic acid, DHBA; Dopamine, DA; Homovanillic acid, HVA; Hydroxyl radical, OH; Parkinson's disease, PD; Phosphodiesterase, PDE; Serotonin, 5-HT; Substantia nigra pars compacta, SNpc

What is claimed is:

1. A method of treating a neurodegenerative disorder comprising:
administering to a subject in need thereof an effective amount of an antioxidant of Formula I or a pharmaceutically acceptable salt thereof 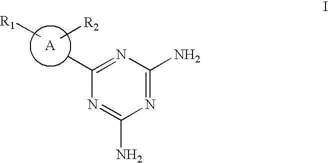
where A is an aromatic ring of 5 or 6 carbons, optionally substituted on an annular carbon with N, S or O;
R₁ and R₂ are independently selected from H, F, Cl, I, Br, C1-6 alkoxy or substituted alkoxy groups, where substituents are selected from amino or substituted amino wherein the substituents are 1-4 carbon alkyl, 3-6 carbon cycloalkyl, or the amino group may be part of a ring containing 3-6 carbon atoms.

2. The method of claim 1, wherein the antioxidant has the structure set forth in Formula II 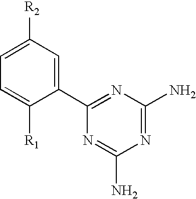
where R₁ and R₂ are independently selected from H, F, Cl, I, and Br.

3. The method of claim 1, wherein said administering is performed for a time and under conditions sufficient to decrease catalepsy or akinesia.

4. The method of claim 1, wherein said administering decreases striatal dopamine depletion.

5. The method of claim 1, wherein said administering decreases production of OH radicals in striatal neurons.

6. The method of claim 1, wherein the subject is an animal model for Parkinson's disease.

7. The method of claim 1, wherein the subject is a human.

8. The method of claim 1, further comprising administering said antioxidant in combination with one or more antipsychotic, antidepressant or serotonin receptor inhibitor.

9. The method of claim 2, wherein the antioxidant is irsogladine, 2,4-diamino-6-(2,5-dichlorophenyl)-s-triazine, or a pharmaceutically acceptable salt thereof.

10. A composition for use in the method of claim 1.

11. The use of an antioxidant of formula I in the manufacture of a medicament for a method according to claim 1.

12. The method of claim 1, wherein said neurodegenerative disorder is striatal neurodegeneration.

13. The method of claim 1, wherein said neurodegenerative disorder is Parkinson's disease or Huntington's disease.

14. The method of claim 1, wherein said treating further comprises administering levodopa in combination with said antioxidant.
